(19) United States
(12) Patent Application Publication
(10) Pub. No.: US 2024/0208920 A1
(43) Pub. Date: Jun. 27, 2024

(54) SYNTHESIS OF MDA OR ITS OPTICALLY ACTIVE (R)- OR (S)-MDA ISOMERS

(71) Applicant: EMPATHBIO, INC., ENCINITAS, CA (US)

(72) Inventors: Majed Fawaz, Foxborough, MA (US); Nicholas Morra, Ontario (CA)

(21) Appl. No.: 18/504,848

(22) Filed: Nov. 8, 2023

Related U.S. Application Data
(60) Provisional application No. 63/423,597, filed on Nov. 8, 2022.

(51) Int. Cl. C07D 317/58

(52) U.S. Cl. CPC C07D 317/58

(57) ABSTRACT

Provided herein is a process for the preparation of 3,4-methylenedioxyamphetamine, (R)-3,4-methylenedioxyamphetamine and (S)-3,4-methylenedioxyamphetamine.

### BACKGROUND

MDA (3,4-methylenedioxyamphetamine) is considered the prototype of a class of compounds called entactogens, which means “to touch within”, their main characteristic being their ability to increase feelings of love, empathy and closeness towards others. Structurally, MDA is a ring-substituted phenethylamine with a chiral molecular center that gives rise to two stereoisomers: S-(+)-MDA and R-(−)-MDA. Typically, effects of the former resemble those of psychostimulants and are primarily mediated by dopaminergic and noradrenergic pathways, including increases in motor activity and euphoria, whereas the latter induces qualitative effects similar to classical psychedelics, such as ego-dissolution and perceptive alterations, mediated by serotonergic pathways, including direct 5-HT2A receptor agonism. The molecular mechanisms for these differences are supported by preclinical evidence and point to a higher therapeutic index for the R-enantiomer.

There remains a need for improved processes of making MDA (3,4-methylenedioxyamphetamine) and pharmaceutically acceptable salts thereof and enantiomers thereof.

### SUMMARY

In an aspect, the present disclosure provides a process for the preparation of 3,4-methylenedioxyamphetamine (MDA). In some embodiments, the present disclosure provides a process for the preparation of racemic 3,4-methylenedioxyamphetamine (MDA). In some embodiments, the present disclosure provides a process for the preparation of enantiopure (R) or (S) 3,4-methylenedioxyamphetamine (MDA).

In embodiments, the present disclosure provides: a process for the preparation of 3,4-methylenedioxyamphetamine, or a pharmaceutically acceptable salt thereof, the process comprising:
i) reacting an organometallic reagent prepared from a compound of Formula (I):

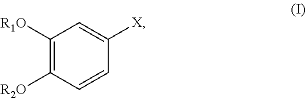

with a compound of Formula (IIb):

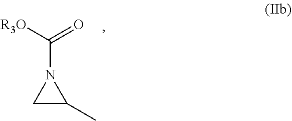

wherein R₃ is alkyl; and
ii) converting the product of step i) to 3,4-methylenedioxyamphetamine, wherein X is a halogen; R₁ is a protecting group, R₂ is a protecting group or R₁ and R₂ together with the atoms to which they are attached form a 5-membered heterocycle.

In embodiments, the compound of Formula (IIb) is racemic.

In embodiments, the present disclosure provides: a process for the preparation of (S)-3,4-methylenedioxyamphetamine, or a pharmaceutically acceptable salt thereof, the process comprising:
i) reacting an organometallic reagent prepared from a compound of Formula (I):

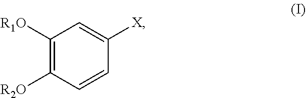

with a compound of Formula (II):

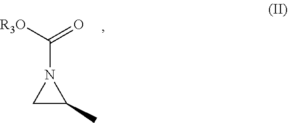

wherein R₃ is alkyl; and
ii) converting the product of step i) to (S)-3,4-methylenedioxyamphetamine wherein X is a halogen; R₁ is a protecting group, R₂ is a protecting group or R₁ and R₂ together with the atoms to which they are attached form a 5-membered heterocycle.

In some embodiments, the present disclosure provides: a process for the preparation of (R)-3,4-methylenedioxyamphetamine, or a pharmaceutically acceptable salt thereof, the process comprising:
i) reacting an organometallic reagent prepared from a compound of Formula (I):

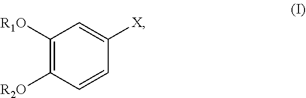

with a compound of Formula (IIa):

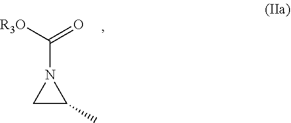

wherein R₃ is alkyl; and
ii) converting the product of step ii) to (R)-3,4-methylenedioxyamphetamine, wherein X is a halogen; R₁ is a protecting group, R₂ is a protecting group or R₁ and R₂ together with the atoms to which they are attached form a 5-membered heterocycle.

In some embodiments, X is preferably bromine.

In some embodiments, the process provides 3,4-methylenedioxyamphetamine, or a pharmaceutically acceptable salt in as a racemate.

In some embodiments, the process provides (S)-3,4-methylenedioxyamphetamine, or a pharmaceutically acceptable salt in an enantiomeric excess of a least 99.5%.

In some embodiments, the process provides (R)-3,4-methylenedioxyamphetamine, or a pharmaceutically acceptable salt in an enantiomeric excess of a least 99.5%.

### DETAILED DESCRIPTION

Throughout this disclosure, various patents, patent applications and publications are referenced. The disclosures of these patents, patent applications and publications in their entireties are incorporated into this disclosure by reference for all purposes in order to more fully describe the state of the art as known to those skilled therein as of the date of this disclosure. This disclosure will govern in the instance that there is any inconsistency between the patents, patent applications and publications cited and this disclosure.

### Definitions

For convenience, certain terms employed in the specification, examples and claims are collected here. Unless defined otherwise, all technical and scientific terms used in this disclosure have the same meanings as commonly understood by one of ordinary skill in the art to which this disclosure belongs.

The term “pharmaceutically acceptable salts” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. The acids that may be used to prepare pharmaceutically acceptable acid addition salts of such basic compounds are those that form non-toxic acid addition salts, i.e., salts containing pharmaceutically acceptable anions, including but not limited to malate, oxalate, chloride, bromide, iodide, nitrate, acetate, tartrate, oleate, fumarate, formate, benzoate, glutamate, methanesulfonate, benzenesulfonate, and p-toluenesulfonate salts. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e.g., lysine and arginine dicyclohexylamine and the like. Examples of metal salts include lithium, sodium, potassium, magnesium, calcium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Those skilled in the art will further recognize that acid addition salts may be prepared by reaction of the compounds with the appropriate inorganic or organic acid via any of a number of known methods.

When a range of values is listed, it is intended to encompass each value and sub-range within the range. For example, “C₁-C₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

“Alkyl” or “alkyl group” refers to a fully saturated, straight or branched hydrocarbon chain having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Heterocyclyl,” “heterocyclic ring” or “heterocycle” refers to a stable saturated, unsaturated, or aromatic 3- to 20-membered ring which consists of two to nineteen carbon atoms and from one to six heteroatoms selected from the group consisting of nitrogen, oxygen and sulfur, and which is attached to the rest of the molecule by a single bond. Heterocyclyl or heterocyclic rings include heteroaryls, heterocyclylalkyls, heterocyclylalkenyls, and hetercyclylalkynyls. Unless stated otherwise specifically in the specification, the heterocyclyl can be a monocyclic, bicyclic, tricyclic or tetracyclic ring system, which can include fused, bridged, or spirocyclic ring systems; and the nitrogen, carbon or sulfur atoms in the heterocyclyl can be optionally oxidized; the nitrogen atom can be optionally quaternized; and the heterocyclyl can be partially or fully saturated. Examples of such heterocyclyl include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

The term “substituted” used herein means any of the groups described herein (e.g., alkyl, heterocyclyl, and/or heteroaryl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups. “Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ. “Substituted” also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl. “Substituted” further means any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group. In addition, each of the foregoing substituents can also be optionally substituted with one or more of the above substituents.

As used herein, the symbol

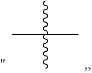

(hereinafter can be referred to as “a point of attachment bond”) denotes a bond that is a point of attachment between two chemical entities, one of which is depicted as being attached to the point of attachment bond and the other of which is not depicted as being attached to the point of attachment bond. For example,

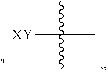

indicates that the chemical entity “XY” is bonded to another chemical entity via the point of attachment bond. Furthermore, the specific point of attachment to the non-depicted chemical entity can be specified by inference. For example, the compound CH₃—R^{3X}, wherein R^{3X} is H or

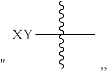

infers that when R^{3X} is “XY”, the point of attachment bond is the same bond as the bond by which R^{3X} is depicted as being bonded to CH₃.

### Methods

In an aspect, the methods described herein provide high purity, 3,4-methylenedioxyamphetamine (MDA) in a high yielding 2-step process, starting from readily available and inexpensive starting materials (e.g., 5-bromobenzo[d][1,3]dioxole (3) and alaninol). In an aspect, the disclosure provides a process for preparation of racemic 3,4-methylenedioxyamphetamine or a pharmaceutically acceptable salt thereof.

In an aspect, the disclosure provides a process for the preparation of (R)- or (S)-3,4-methylenedioxyamphetamine, or a pharmaceutically acceptable salt thereof.

In embodiments, the methods described herein provide high purity, enantiopure (R) or (S) 3,4-methylenedioxyamphetamine (MDA) in a high yielding 2-step process, starting from readily available and inexpensive starting materials (e.g., 5-bromobenzo[d][1,3]dioxole (3) and D-alaninol (1)). By starting with a chiral pool starting materials, such as (D-alaniol (1)), enantiopure (R)-MDA can be prepared without the need for expensive and wasteful chiral ligands, chiral auxiliaries, or diastereomeric salt resolutions, and provide MDA with higher optical purity (e.g., 99.5% ee or greater, or 99.9% ee) than other routes, which give lower selectivity and require enantiomeric enrichment by purification/crystallization. By installing a Boc group as a nitrogen protecting group, the reaction avoids the potential of over-alkylation and provides enantioenriched (R)-MDA in a streamlined process, providing significant improvements on prior syntheses which are more complex and/or require synthetic longer routes see e.g., ACS Chem Neurosci. 2018 October 17; 9(10): 2408-2427, FIG. 3. The unnatural enantiomer of (1) (L-alaninol) can be used by the same process to prepare (S)-MDA.

### Preparation of 3,4-Methylenedioxyamphetamine

In an aspect, the disclosure provides a process for the preparation of 3,4-methylenedioxyamphetamine, or a pharmaceutically acceptable salt thereof, the process comprising:
i) reacting an organometallic reagent prepared from a compound of Formula (I):

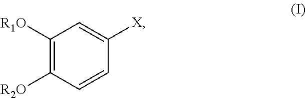

with a compound of Formula (IIb):

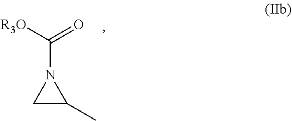

wherein R₃ is alkyl; and
ii) converting the product of step i) to 3,4-methylenedioxyamphetamine, wherein X is a halogen; R₁ is a protecting group, R₂ is a protecting group or R₁ and R₂ together with the atoms to which they are attached form a 5-membered heterocycle.

In embodiments, the compound of Formula (IIb) is a racemate.

In embodiments of the processes provided herein for the preparation of 3,4-methylenedioxyamphetamine, the process comprises i) preparing an organometallic reagent from a compound of Formula (I):

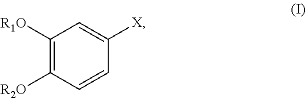

wherein X is a halogen; R₁ is a protecting group, R₂ is a protecting group or R₁ and R₂ together with the atoms to which they are attached form a 5-membered heterocycle.

In some embodiments of any one of the processes for the preparation of 3,4-methylenedioxyamphetamine described herein, R₁ and R₂ together with the atoms to which they are attached form a 5-membered heterocycle.

In some embodiments of the process for the preparation of 3,4-methylenedioxyamphetamine, the compound of Formula (I) is a compound of Formula (Ia):

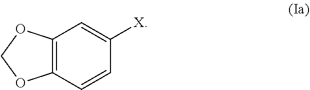

In embodiments of the compounds of Formula (I) or (Ia), X is Cl, Br, or I. In some embodiments, X is Cl. In some embodiments, X is Br. In some embodiments, X is I.

In some embodiments of the process for the preparation of 3,4-methylenedioxyamphetamine, step i) comprises reacting the compound of Formula (I) or Formula (Ia) with magnesium. In some embodiments, step i) comprises reacting the compound of Formula (I) or Formula (Ia) with magnesium in the presence of a solvent, for example an ether solvent such as tetrahydrofuran, diethyl ether, or 2-methyltetrahydrofuran. In some embodiments, the solvent is heated e.g., to 50-70° C., 60-70° C., or 60-66° C. In some embodiments, the solvent is heated to reflux. In some embodiments, the solvent is THF and the THF is heated to reflux.

In some embodiments of the process for the preparation of 3,4-methylenedioxyamphetamine, step i) further comprises adding a copper (I) salt (e.g., CuI, CuCl, or CuBr·SMe₂) to the reaction mixture. In some embodiments, after formation of the Grignard species, the solution is cooled, and copper iodide (CuI) is added. In some embodiments, the solution is cooled for example, to a temperature between 0° C. and −78° C. and copper iodide (CuI) is added. In some embodiments, the organometallic reagent selectively opens the aziridine at the less hindered carbon, retaining the stereochemistry of the nitrogen stereocenter to give carbamate protected methylenedioxyamphetamine.

In embodiments of the process for the preparation of 3,4-methylenedioxyamphetamine, step i) comprises reacting the organometallic reagent of step i) with a compound of Formula (IIb):

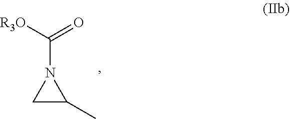

wherein R₃ is alkyl. In some embodiments, step i) comprises adding a compound of Formula (IIb) to the cooled solution of organometallic reagent.

In some embodiments of the process for the preparation of 3,4-methylenedioxyamphetamine, R₃ is C₁₋₆ alkyl. In some embodiments, R₃ is C₁₋₄ alkyl. In some embodiments, R₃ is tert-butyl.

In some embodiments of the process for the preparation of 3,4-methylenedioxyamphetamine, the product of step i) is a compound of Formula (IIIb):

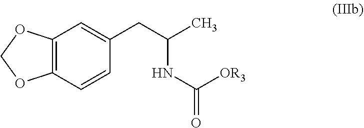

wherein R₃ is defined herein.

In embodiments of the process for the preparation of 3,4-methylenedioxyamphetamine, the product of step i) is a compound of Formula (IIIc):

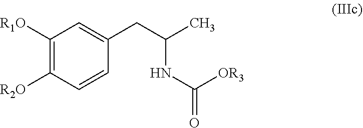

wherein R₁, R₂, and R₃ are defined herein.

In some embodiments of the process for the preparation of 3,4-methylenedioxyamphetamine, step ii) comprises reacting a group of Formula (IVb) with a deprotecting agent to provide a group of Formula (Vb), or a pharmaceutically acceptable salt thereof:

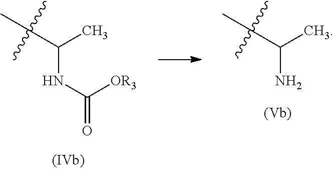

In some embodiments, step ii) comprises reacting a group of Formula (IVb) with a deprotecting agent to provide a group of Formula (Vb), or a pharmaceutically acceptable salt thereof in the presence of a solvent, for example an ether solvent such as tetrahydrofuran, diethyl ether, 2-methyltetrahydrofuran. In some embodiments, the solvent is heated. In some embodiments, the solvent is refluxing THF.

In some embodiments of the process for the preparation of 3,4-methylenedioxyamphetamine, the deprotecting agent in step ii) is an acid. In some embodiments, the deprotecting agent in step ii) is hydrochloric acid.

In some embodiments of the process for the preparation of 3,4-methylenedioxyamphetamine, the process provides 3,4-methylenedioxyamphetamine, or a pharmaceutically acceptable salt thereof in racemic form.

In some embodiments of the process for the preparation of 3,4-methylenedioxyamphetamine, the 3,4-methylenedioxyamphetamine, or a pharmaceutically acceptable salt thereof has a chemical purity of greater than 95%, greater than 98%, or greater than 99% by HPLC.

In some embodiments, the present disclosure provides 3,4-methylenedioxyamphetamine, or a pharmaceutically acceptable salt prepared by a process described herein.

### Preparation of (S)-3,4-Methylenedioxyamphetamine

In an aspect, the disclosure provides a process for the preparation of (S)-3,4-methylenedioxyamphetamine, or a pharmaceutically acceptable salt thereof, the process comprising:
i) reacting an organometallic reagent prepared from a compound of Formula (I):

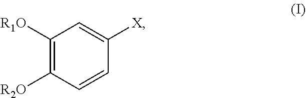

with a compound of Formula (II):

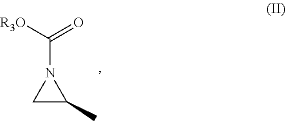

wherein R₃ is alkyl; and
ii) converting the product of step ii) to (S)-3,4-methylenedioxyamphetamine, wherein X is a halogen; R₁ is a protecting group, R₂ is a protecting group or R₁ and R₂ together with the atoms to which they are attached form a 5-membered heterocycle.

In embodiments of the processes provided herein for the preparation of (S)-3,4-methylenedioxyamphetamine, the process comprises preparing an organometallic reagent from a compound of Formula (I):

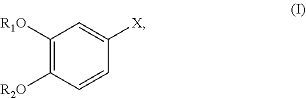

wherein X is a halogen; R₁ is a protecting group, R₂ is a protecting group or R₁ and R₂ together with the atoms to which they are attached form a 5-membered heterocycle.

In some embodiments of any one of the processes for the preparation of (S)-3,4-methylenedioxyamphetamine described herein, R₁ and R₂ together with the atoms to which they are attached form a 5-membered heterocycle.

In some embodiments of the process for the preparation of (S)-3,4-methylenedioxyamphetamine, the compound of Formula (I) is a compound of Formula (Ia):

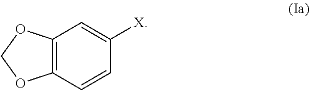

In embodiments of the compounds of Formula (I) or (Ia), X is Cl, Br, or I. In some embodiments, X is Cl. In some embodiments, X is Br. In some embodiments, X is I.

In some embodiments of the process for the preparation of (S)-3,4-methylenedioxyamphetamine, step i) comprises reacting the compound of Formula (I) or Formula (Ia) with magnesium. In some embodiments, step i) comprises reacting the compound of Formula (I) or Formula (Ia) with magnesium in the presence of a solvent, for example an ether solvent such as tetrahydrofuran, diethyl ether, 2-methyltetrahydrofuran. In some embodiments, the solvent is heated e.g., to 50-70° C., or 60-70° C., or 60-66° C. In some embodiments, the solvent is heated to reflux. In some embodiments, the solvent is THF and the THF is heated to reflux.

In some embodiments of the process for the preparation of (S)-3,4-methylenedioxyamphetamine, step i) further comprises adding a copper (I) salt (e.g., CuI, CuCl, CuBr·SMe₂) to the reaction mixture. In some embodiments, after formation of the Grignard species, the solution is cooled, and copper iodide (CuI) is added. In some embodiments, the solution is cooled for example, to a temperature between 0° C. and −78° C. and copper iodide (CuI) is added. In some embodiments, the organometallic reagent selectively opens the aziridine at the less hindered carbon, retaining the stereochemistry of the nitrogen stereocenter to give carbamate protected methylenedioxyamphetamine.

In embodiments of the process for the preparation of (S)-3,4-methylenedioxyamphetamine, step i) further comprises reacting the organometallic reagent with a compound of Formula (II):

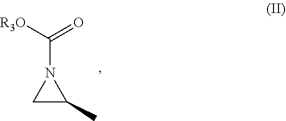

wherein R₃ is alkyl. In some embodiments, step ii) comprises adding a compound of Formula (II) to the cooled solution of organometallic reagent.

In some embodiments of the process for the preparation of (S)-3,4-methylenedioxyamphetamine, R₃ is C₁₋₆ alkyl. In some embodiments, R₃ is C₁₋₄ alkyl. R₃ is tert-butyl.

In some embodiments of the process for the preparation of (S)-3,4-methylenedioxyamphetamine, the product of step i) is a compound of Formula (III):

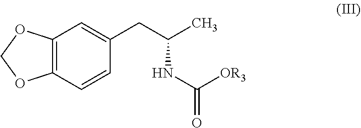

wherein R₃ is defined herein.

In some embodiments of the process for the preparation of (S)-3,4-methylenedioxyamphetamine, the product of step i) is a compound of Formula (IIIa):

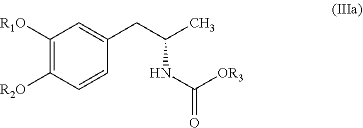

wherein R₁, R₂, and R₃ are defined herein.

In some embodiments of the process for the preparation of (S)-3,4-methylenedioxyamphetamine, step ii) comprises reacting a group of Formula (IV) with a deprotecting agent to provide a group of Formula (V), or a pharmaceutically acceptable salt thereof:

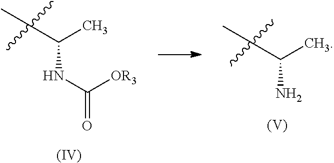

In some embodiments, step ii) comprises reacting a group of Formula (IV) with a deprotecting agent to provide a group of Formula (V), or a pharmaceutically acceptable salt thereof in the presence of a solvent, for example an ether solvent such as tetrahydrofuran, diethyl ether, 2-methyltetrahydrofuran. In some embodiments, the solvent is heated. In some embodiments, the solvent is refluxing THF.

In some embodiments of the process for the preparation of (S)-3,4-methylenedioxyamphetamine, the deprotecting agent in step ii) is an acid. In some embodiments, the deprotecting agent in step ii) is hydrochloric acid.

In some embodiments of the process for the preparation of (S)-3,4-methylenedioxyamphetamine, the process provides (S)-3,4-methylenedioxyamphetamine, or a pharmaceutically acceptable salt thereof in substantially optically pure form. In some embodiments, the process for the preparation of (S)-3,4-methylenedioxyamphetamine, the process provides (S)-3,4-methylenedioxyamphetamine, or a pharmaceutically acceptable salt in an enantiomeric excess (ee) of about or at least about 55% ee, about or at least about 60% ee, about or at least about 65% ee, about or at least about 70% ee, about or at 75% ee, about or at least about 80% ee, about or at least about 85% ee, about or at least about 90% ee, about or at least about 91%, about or at least about 92%, about or at least about 93% ee, about or at least about 94% ee, about or at least about 95% ee, about or at least about 96% ee, about or at least about 97% ee, about or at least about 98% ee, about or at least about 99% ee, about or at least about 99.5% ee, or about or at least about 99.9% ee, including all subranges and values therebetween. In some embodiments, the process for the preparation of (S)-3,4-methylenedioxyamphetamine, the process provides (S)-3,4-methylenedioxyamphetamine, or a pharmaceutically acceptable salt in an enantiomeric excess of a least 99.5%. In some embodiments, ee is measured by chiral HPLC.

In some embodiments of the process for the preparation of (S)-3,4-methylenedioxyamphetamine, the (S)-3,4-methylenedioxyamphetamine, or a pharmaceutically acceptable salt thereof has a chemical purity of greater than 95%, greater than 98%, or greater than 99% by HPLC.

In some embodiments, the present disclosure provides (S)-3,4-methylenedioxyamphetamine, or a pharmaceutically acceptable salt prepared by a process described herein.

### Preparation of (R)-3,4-Methylenedioxyamphetamine

In an aspect, the disclosure provides a process or the preparation of (R)-3,4-methylenedioxyamphetamine, or a pharmaceutically acceptable salt thereof, the method comprising:
i) reacting an organometallic reagent prepared from a compound of Formula (I):

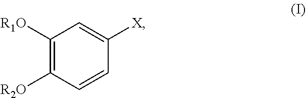

with a compound of Formula (IIa):

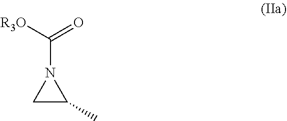

wherein R₃ is alkyl; and
ii) converting the product of step ii) to (R)-3,4-methylenedioxyamphetamine, wherein X is a halogen; R₁ is a protecting group, R₂ is a protecting group or R₁ and R₂ together with the atoms to which they are attached form a 5-membered heterocycle.

In embodiments of the processes provided herein for the preparation of (R)-3,4-methylenedioxyamphetamine, the process comprises i) preparing an organometallic reagent from a compound of Formula (I):

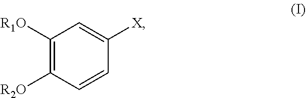

wherein X is a halogen; R₁ is a protecting group, R₂ is a protecting group or R₁ and R₂ together with the atoms to which they are attached form a 5-membered heterocycle.

In some embodiments of any one of the processes for the preparation of (R)-3,4-methylenedioxyamphetamine described herein, R₁ and R₂ together with the atoms to which they are attached form a 5-membered heterocycle.

In some embodiments of the process for the preparation of (R)-3,4-methylenedioxyamphetamine, the compound of Formula (I) is a compound of Formula (Ia):

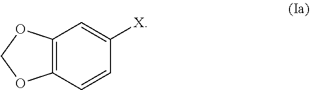

In embodiments of the compounds of Formula (I) or (Ia), X is Cl, Br, or I. In some embodiments, X is Cl. In some embodiments, X is Br. In some embodiments, X is I.

In some embodiments of the process for the preparation of (R)-3,4-methylenedioxyamphetamine, step i) comprises reacting the compound of Formula (I) or Formula (Ia) with magnesium. In some embodiments, step i) comprises reacting the compound of Formula (I) or Formula (Ia) with magnesium in the presence of a solvent, for example an ether solvent such as tetrahydrofuran, diethyl ether, 2-methyltetrahydrofuran. In some embodiments, the solvent is heated e.g., to 50-70° C. In some embodiments, the solvent is THF and the THF is heated to reflux.

In some embodiments of the process for the preparation of (R)-3,4-methylenedioxyamphetamine, step i) further comprises adding a copper (I) salt (e.g., CuI, CuCl, CuBr·SMe₂) to the reaction mixture. In some embodiments, after formation of the Grignard species, the solution is cooled, and copper iodide (CuI) is added. In some embodiments, the solution is cooled for example, to a temperature between 0° C.and −78° C. and copper iodide (CuI) is added. In some embodiments, the organometallic reagent selectively opens the aziridine at the less hindered carbon, retaining the stereochemistry of the nitrogen stereocenter to give carbamate protected methylenedioxyamphetamine.

In some embodiments of the process for the preparation of (R)-3,4-methylenedioxyamphetamine, comprises reacting the organometallic reagent with a compound of Formula (IIa):

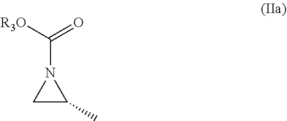

wherein R₃ is alkyl. In some embodiments, step ii) comprises adding a compound of Formula (IIa) to the cooled solution of organometallic reagent from step i).

In some embodiments of the process for the preparation of (R)-3,4-methylenedioxyamphetamine, R₃ is C₁₋₆ alkyl. In some embodiments, R₃ is C₁₋₄ alkyl. R₃ is tert-butyl.

In some embodiments of the process for the preparation of (R)-3,4-methylenedioxyamphetamine, the product of step i) is a compound of Formula (IIIb):

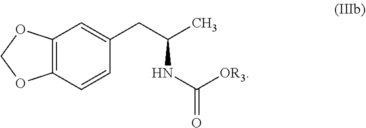

In some embodiments of the process for the preparation of (R)-3,4-methylenedioxyamphetamine, the product of step i) is a compound of Formula (IIIb′):

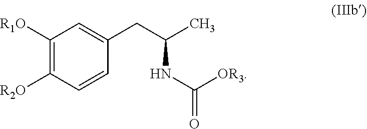

In some embodiments of the process for the preparation of (R)-3,4-methylenedioxyamphetamine, step ii) comprises reacting a group of Formula (IVa) with a deprotecting agent to provide a group of Formula (Va), or a pharmaceutically acceptable salt thereof:

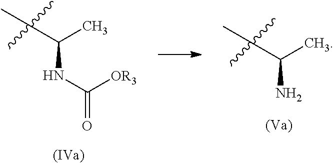

In some embodiments, step ii) comprises reacting a group of Formula (IV) with a deprotecting agent to provide a group of Formula (V), or a pharmaceutically acceptable salt thereof in the presence of a solvent, for example an ether solvent such as tetrahydrofuran, diethyl ether, 2-methyltetrahydrofuran.

In some embodiments of the process for the preparation of (R)-3,4-methylenedioxyamphetamine, the deprotecting agent in step ii) is an acid. In some embodiments, the deprotecting agent in step ii) is hydrochloric acid.

In some embodiments of the process for the preparation of (R)-3,4-methylenedioxyamphetamine, the process provides (R)-3,4-methylenedioxyamphetamine, or a pharmaceutically acceptable salt thereof in substantially optically pure form. In some embodiments, the process for the preparation of (R)-3,4-methylenedioxyamphetamine, the process provides (R)-3,4-methylenedioxyamphetamine, or a pharmaceutically acceptable salt in an enantiomeric excess (ee) of about or at least about 55% ee, about or at least about 60% ee, about or at least about 65% ee, about or at least about 70% ee, about or at 75% ee, about or at least about 80% ee, about or at least about 85% ee, about or at least about 90% ee, about or at least about 91%, about or at least about 92%, about or at least about 93% ee, about or at least about 94% ee, about or at least about 95% ee, about or at least about 96% ee, about or at least about 97% ee, about or at least about 98% ee, about or at least about 99% ee, about or at least about 99.5% ee, or about or at least about 99.9% ee, including all subranges and values therebetween. In some embodiments, the process for the preparation of (R)-3,4-methylenedioxyamphetamine, the process provides (R)-3,4-methylenedioxyamphetamine, or a pharmaceutically acceptable salt in an enantiomeric excess of a least 99.5%. In some embodiments, ee is measured by chiral HPLC.

In some embodiments of the process for the preparation of (R)-3,4-methylenedioxyamphetamine, the (R)-3,4-methylenedioxyamphetamine, or a pharmaceutically acceptable salt thereof has a chemical purity of greater than 95%, greater than 98%, or greater than 99% by HPLC.

In some embodiments, the present disclosure provides (R)-3,4-methylenedioxyamphetamine, or a pharmaceutically acceptable salt prepared by a process described herein.

### Numbered Embodiments

1. A process for the preparation of (S)-3,4-methylenedioxyamphetamine, or a pharmaceutically acceptable salt thereof, the process comprising:
i) reacting an organometallic reagent prepared from a compound of Formula (I):

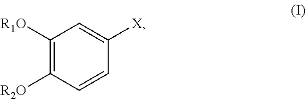

with a compound of Formula (II):

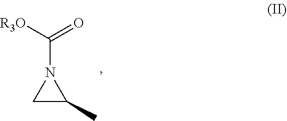

wherein R₃ is alkyl; and
ii) converting the product of step ii) to (S)-3,4-methylenedioxyamphetamine, wherein X is a halogen; R₁ is a protecting group, R₂ is a protecting group or R₁ and R₂ together with the atoms to which they are attached form a 5-membered heterocycle.

2. The process of embodiment 1, wherein the compound of Formula (I) is a compound of Formula (Ia):

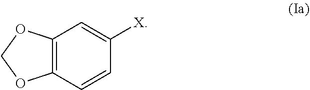

3. The process of embodiment 2, wherein X is bromine.

4. The process of any one of embodiments 1-3, wherein R₃ is tert-butyl.

5. The process of any one of embodiments 1-4, wherein step i) comprises reacting the compound of Formula (I) or Formula (Ia) with magnesium.

6. The process of embodiment 5, wherein the step i) further comprises adding a copper (I) salt (e.g., CuI) to the reaction mixture.

7. The process of any one of embodiments 1-6, wherein the product of step i) is a compound of Formula (III):

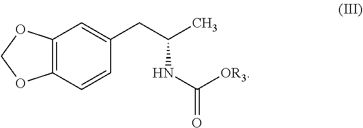

8. The process of any one of embodiments 1-6, wherein the product of step i) is a compound of Formula (IIIa):

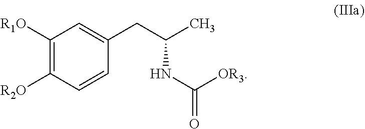

9. The process of any one of embodiments 1-8, wherein the step ii) comprises reacting a group of Formula (IV) with a deprotecting agent to provide a group of Formula (V), or a pharmaceutically acceptable salt thereof:

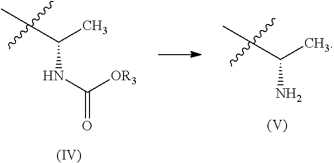

10. The process of embodiment 9, wherein the deprotecting agent is hydrochloric acid.

11. The process of any one of embodiments 1-10, wherein the process provides (S)-3,4-methylenedioxyamphetamine, or a pharmaceutically acceptable salt in an enantiomeric excess of a least 99.5%.

12. (S)-3,4-methylenedioxyamphetamine, or a pharmaceutically acceptable salt prepared by a process of any one of embodiments 1-10.

13. A process for the preparation of (R)-3,4-methylenedioxyamphetamine, or a pharmaceutically acceptable salt thereof, the process comprising:
i) reacting an organometallic reagent prepared from a compound of Formula (I):

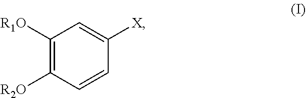

with a compound of Formula (IIa):

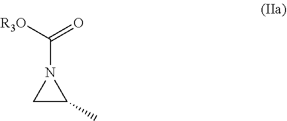

wherein R₃ is alkyl; and
ii) converting the product of step ii) to (R)-3,4-methylenedioxyamphetamine, wherein X is a halogen; R₁ is a protecting group, R₂ is a protecting group or R₁ and R₂ together with the atoms to which they are attached form a 5-membered heterocycle.

14. The process of embodiment 13, wherein the compound of Formula (I) is a compound of Formula (Ia):

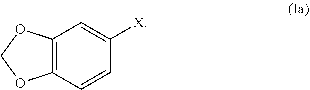

15. The process of any one of embodiments 13-14, wherein X is bromine.

16. The process of any one of embodiments 13-15, wherein R₃ is tert-butyl.

17. The process of any one of embodiments 13-16, wherein step i) comprises reacting the compound of Formula (I) or Formula (Ia) with magnesium.

18. The process of embodiment 17, wherein the step i) further comprises adding a copper (I) salt (e.g., CuI) to the reaction mixture.

19. The process of any one of embodiments 13-18, wherein the product of step i) is a compound of Formula (IIIb):

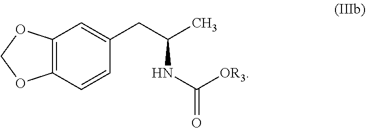

20. The process of any one of embodiments 13-18, wherein the product of step i) is a compound of Formula (IIIb′):

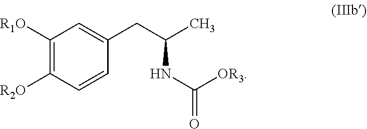

21. The process of any one of embodiments 13-20, wherein the step ii) comprises reacting a group of Formula (IVa) with a deprotecting agent to provide a group of Formula (Va), or a pharmaceutically acceptable salt thereof:

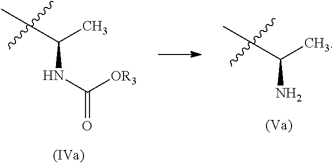

22. The process of embodiment 21, wherein the deprotecting agent is hydrochloric acid.

23. The process of any one of embodiments 13-22, wherein the (R)-3,4-methylenedioxyamphetamine, or pharmaceutically acceptable salt is in an enantiomeric excess of a least 99.5%.

24. (R)-3,4-methylenedioxyamphetamine, or a pharmaceutically acceptable salt prepared by a process of any one of embodiments 13-23.

25. A process for the preparation of 3,4-methylenedioxyamphetamine, or a pharmaceutically acceptable salt thereof, the process comprising:
i) reacting an organometallic reagent prepared from a compound of Formula (I):

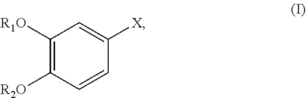

with a compound of Formula (II):

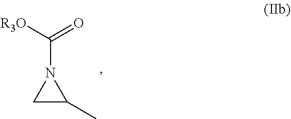

wherein R₃ is alkyl; and
ii) converting the product of step ii) to 3,4-methylenedioxyamphetamine, wherein X is a halogen; R₁ is a protecting group, R₂ is a protecting group or R₁ and R₂ together with the atoms to which they are attached form a 5-membered heterocycle.

26. The process of embodiment 25, wherein the compound of Formula (I) is a compound of Formula (Ia):

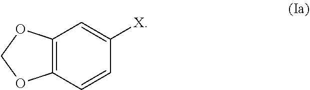

27. The process of embodiment 26, wherein X is bromine.

28. The process of any one of embodiments 25-27, wherein R₃ is tert-butyl.

29. The process of any one of embodiments 25-28, wherein step i) comprises reacting the compound of Formula (I) or Formula (Ia) with magnesium.

30. The process of embodiment 29, wherein the step i) further comprises adding a copper (I) salt (e.g., CuI) to the reaction mixture.

31. The process of any one of embodiments 25-30, wherein the product of step i) is a compound of Formula (III):

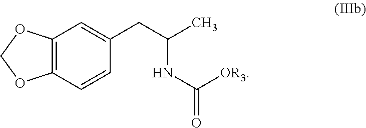

32. The process of any one of embodiments 25-30, wherein the product of step i) is a compound of Formula (IIIc):

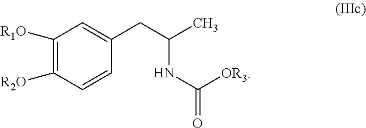

33. The process of any one of embodiments 1-8, wherein the step ii) comprises reacting a group of Formula (IVb) with a deprotecting agent to provide a group of Formula (Vb), or a pharmaceutically acceptable salt thereof:

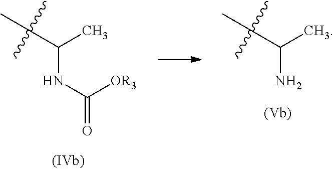

34. The process of embodiment 33, wherein the deprotecting agent is hydrochloric acid.

35. The process of any one of embodiments 25-34, wherein the (S)-3,4-methylenedioxyamphetamine or pharmaceutically acceptable salt is in an enantiomeric excess of a least 99.5%.

36. 3,4-methylenedioxyamphetamine, or a pharmaceutically acceptable salt prepared by a process of any one of embodiments 25-35.

### EXAMPLES

Compounds of the present disclosure are synthesized using the following exemplary methods or other methods that are known to those skilled in the art.

General reaction conditions are provided, and reaction products are purified by known methods including silica gel chromatography using various organic solvents such as hexane, dichloromethane, ethyl acetate, methanol and the like or preparative reverse phase high pressure liquid chromatography.

Preparation of compounds involves the protection and deprotection of various chemical groups. The need for protection and deprotection, and the selection of appropriate protecting groups is readily determined by one skilled in the art. The chemistry of protecting groups can be found, for example, in Greene and Wuts, Protective Groups in Organic Synthesis, 44th. Ed., Wiley & Sons, 2006, as well as in Jerry March, Advanced Organic Chemistry, 4^{th} edition, John Wiley & Sons, publisher, New York, 1992 which are incorporated herein by reference in their entirety.

### General Synthesis of (R)-3,4-Methylenedioxyamphetamine (MDA)

(R)-3,4-methylenedioxyamphetamine (MDA) is synthesized according to Scheme 1, below.

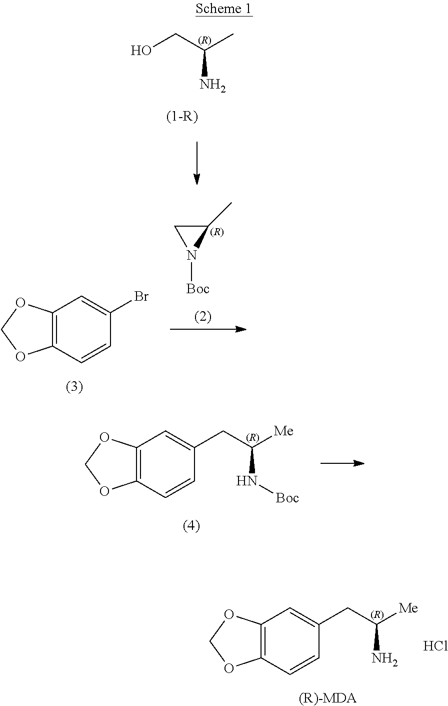

Step 1: Synthesis of tert-butyl (R)-2-methylaziridine-1-carboxylate (2)

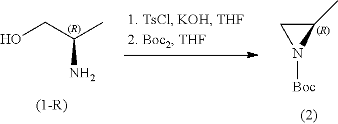

To D-alaninol (1) is added 4-toluenesulfonyl chloride (TsCl), followed by potassium hydroxide (KOH), which installs the tosyl group on the alcohol and deprotonates the nitrogen, facilitating aziridine formation. The enantiopure aziridine intermediate is subsequently protected with di-tert-butyl dicarbonate (Boc anhydride) in organic solvent (e.g., halogenated solvent such as dichloromethane (DCM), either solvent such as tetrahydrofuran (THF) or methyl-tetrahydrofuran (Me-THF)) to form compound (2), which is then purified. In the scheme above, compound (2) may be synthesized as shown or it may be purchased since compound (2) is a commercially available compound.

Step 2: Synthesis of tert-butyl (R)-(1-(benzo[d][1,3]dioxol-5-yl)propan-2-yl)carbamate (4)

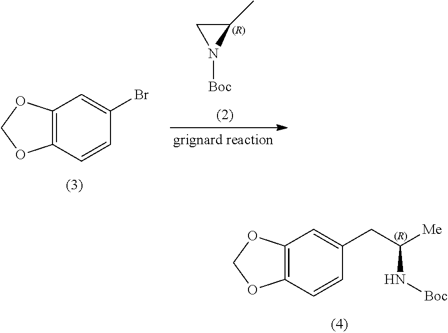

In a second step, the Grignard reagent of 5-bromobenzo[d][1,3]dioxole (3) is generated by treatment of 5-bromobenzo[d][1,3]dioxole (3) with magnesium, optionally in the presence of I₂, in organic solvent (e.g., ether solvent such as tetrahydrofuran (THF) or methyl-tetrahydrofuran (Me-THF)) and the mixture heated. After formation of the Grignard species, the solution is cooled and Copper (I) salt e.g., CuI or CuBr·SMe₂ is added. The previously isolated Boc-aziridine (2) is then added to the cooled solution of cuprate and stirred until the reaction is complete. The reaction is then quenched with an aqueous solution e.g., with NH₄Cl and the organic phase extracted with organic solvent and purified by chromatography on SiO₂, or by crystallization.

### Step 3: Synthesis of (R)-3,4-Methylenedioxyamphetamine (MDA)

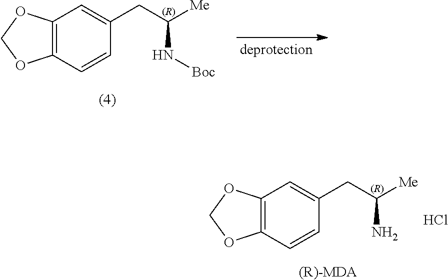

The Boc-MDA (4) is then dissolved in organic solvent (e.g., in an ether solvent such as tetrahydrofuran, (THF) or 2-methyltetrahydrofuran (Me-THF)). The Boc protecting group of compound (4) may be removed under strongly acidic conditions to generate the free amine.

(S)-3,4-methylenedioxyamphetamine (MDA) can be synthesized according to Scheme 1 employing L-alaninol instead of D-alaninol.

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

1. A process for the preparation of (S)-3,4-methylenedioxyamphetamine, or a pharmaceutically acceptable salt thereof, the process comprising:
i) reacting an organometallic reagent prepared from a compound of Formula (I): 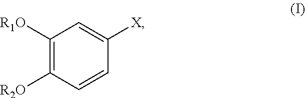
with a compound of Formula (II): 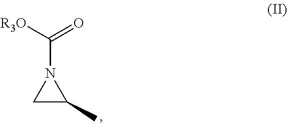
wherein R₃ is alkyl; and
ii) converting the product of step i) to (S)-3,4-methylenedioxyamphetamine, wherein X is a halogen; R₁ is a protecting group, R₂ is a protecting group or R₁ and R₂ together with the atoms to which they are attached form a 5-membered heterocycle.

2. The process of claim 1, wherein the compound of Formula (I) is a compound of Formula (Ia): 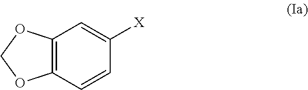
wherein the product of step i) is a compound of Formula (III):

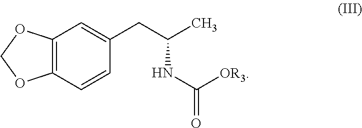

3. The process of claim 1, wherein X is bromine and R₃ is tert-butyl.

4. (canceled)

5. The process of claim 1, wherein step i) comprises reacting the compound of Formula (I) with magnesium.

6. The process of claim 5, wherein the step i) further comprises adding a copper (I) salt to the reaction mixture.

7. (canceled)

8. The process of claim 1, wherein the product of step i) is a compound of Formula (IIIa):

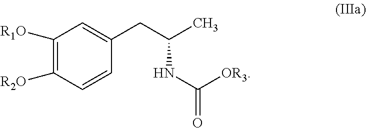

9. The process of claim 1, wherein the step ii) comprises reacting a group of Formula (IV) with a deprotecting agent to provide a group of Formula (V), or a pharmaceutically acceptable salt thereof:

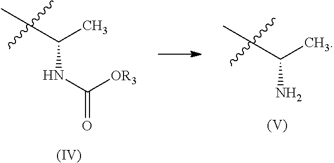

10. (canceled)

11. The process of claim 1, wherein the (S)-3,4-methylenedioxyamphetamine, or pharmaceutically acceptable salt is in an enantiomeric excess of a least 99.5%.

12. (canceled)

13. A process for the preparation of (R)-3,4-methylenedioxyamphetamine, or a pharmaceutically acceptable salt thereof, comprising:
i) reacting an organometallic reagent prepared from a compound of Formula (I): 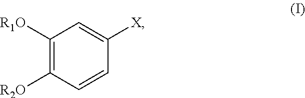
with a compound of Formula (IIa): 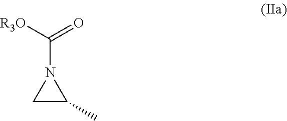
wherein R₃ is alkyl; and
ii) converting the product of step i) to (R)-3,4-methylenedioxyamphetamine, wherein X is a halogen; R₁ is a protecting group, R₂ is a protecting group or R₁ and R₂ together with the atoms to which they are attached form a 5-membered heterocycle;
ii) reacting the organometallic reagent of step i).

14. The process of claim 13, wherein the compound of Formula (I) is a compound of Formula (Ia): 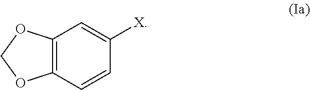
wherein the product of step i) is a compound of Formula (IIIb):

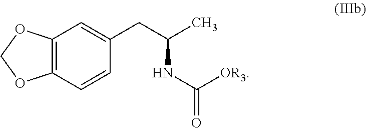

15. The process of claim 13, wherein X is bromine and R₃ is tert-butyl.

16. (canceled)

17. The process of claim 13, wherein step i) comprises reacting the compound of Formula (I) with magnesium.

18. The process of claim 17, wherein the step i) further comprises adding a copper (I) salt to the reaction mixture.

19. (canceled)

20. The process of claim 13, wherein the product of step i) is a compound of Formula (IIIb′):

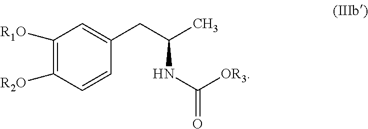

21. The process of 13, wherein the step ii) comprises reacting a group of Formula (IVa) with a deprotecting agent to provide a group of Formula (Va), or a pharmaceutically acceptable salt thereof:

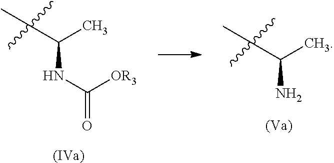

22. (canceled)

23. The process of claim 13, wherein the (R)-3,4-methylenedioxyamphetamine or pharmaceutically acceptable salt is in an enantiomeric excess of a least 99.5%.

24. (canceled)

25. A process for the preparation of 3,4-methylenedioxyamphetamine, or a pharmaceutically acceptable salt thereof, the process comprising:
i) reacting an organometallic reagent prepared from a compound of Formula (I): 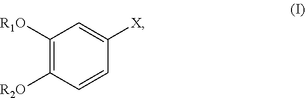
with a compound of Formula (IIb): 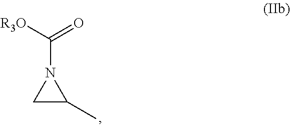
wherein R₃ is alkyl; and
ii) converting the product of step i) to 3,4-methylenedioxyamphetamine, wherein X is a halogen; R₁ is a protecting group, R₂ is a protecting group or R₁ and R₂ together with the atoms to which they are attached form a 5-membered heterocycle.

26. (canceled)

27. The process of claim 25, wherein the product of step i) is a compound of Formula (IIIc):

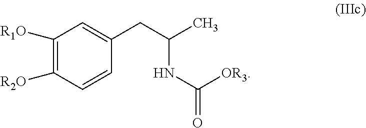

28. The process of claim 25, wherein the step ii) comprises reacting a group of Formula (IVb) with a deprotecting agent to provide a group of Formula (Vb), or a pharmaceutically acceptable salt thereof:

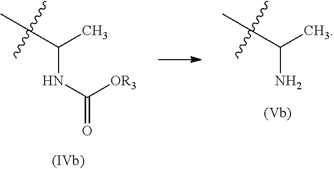

29. (canceled)

30. (canceled)

31. The process of claim 25, wherein the method produces 3,4-methylenedioxyamphetamine, or a pharmaceutically acceptable salt with a purity of at least 99%.

32. (canceled)

33. (canceled)

34. (canceled)

35. (canceled)
